(19) United States
(12) Patent Application Publication
(10) Pub. No.: US 2021/0128520 A1
(43) Pub. Date: May 6, 2021

(54) NEUROPROTECTIVE ISOFLAVONE COMPOSITIONS AND METHODS

(71) Applicants: Ke Jim, Albuquerque, NM (US); Jiangang Shen, Albuquerque, NM (US); UNM Rainforest Innovations, Albuquerque, NM (US)

(72) Inventors: Ke Jian Jim Liu, Albuquerque, NM (US); Jiangang Shen, Hong Kong (CN)

(21) Appl. No.: 16/632,742

(22) PCT Filed: Jul. 24, 2018
(86) PCT No.: PCT/US2018/043442

§ 371 Date: Jan. 21, 2020

Related U.S. Application Data
(60) Provisional application No. 62/536,095, filed on Jul. 24, 2017.

(51) Int. Cl. A61K 31/352; A61K 31/704; A61K 31/198; A61K 31/192; A61P 9/10

(52) U.S. Cl. CPC A61K 31/352; A61K 31/704; A61P 9/10; A61K 31/192; A61K 31/198

(57) ABSTRACT

A composition includes two or more isoflavone compounds and a pharmaceutically acceptable carrier. In some cases, the isoflavone compounds can include calycosin, formononetin, or daidzein. The composition can be used in methods to treat a subject having at risk of having cerebral ischemia-reperfusion injury and/or hypoxia brain injury.

### CROSS-REFERENCE TO RELATED APPLICATION

This application claims priority to U.S. Provisional Patent Application No. 62/536,095, filed Jul. 24, 2017, which is incorporated herein by reference.

### SUMMARY

This disclosure describes, in one aspect, a composition that includes a combination of two or isoflavone compounds and a pharmaceutically-acceptable carrier. The isoflavone compounds include calycosin, formononetin, or daidzein.

In some embodiments, the composition can be provided in an amount effective to decrease infarct volume in a rat model.

In some embodiments, the composition can be provided in an amount effective to decrease neural death by in vivo treatment with L-glutamate.

In some embodiments, the composition can be provided in an amount effective to decrease neural death in an in vivo oxygen-glucose deprivation plus reoxygenation (OGD/RO) model.

In some embodiments, the composition can be provided in an amount effective to decrease hypoxia.

In some embodiments, the composition can include calycosin, formononetin, and daidzein.

In some embodiments, the composition can further include ginsenoside Rg1. In some of these embodiments, the ginsenoside Rg1 can be provided in an amount effective to further decrease infarct volume in a rat model, further decrease neural death by in vivo treatment with L-glutamate, further decrease neural death in an in vivo OGD/RO model, or further decrease hypoxia.

In some embodiments, the composition can further include a salt of danshensu. In some of these embodiments, the salt of danshensu can be provided in an amount effective to further decrease infarct volume in a rat model, further decrease neural death by in vivo treatment with L-glutamate, further decrease neural death in an in vivo OGD/RO model, or further decrease hypoxia.

In another aspect, this disclosure describes a method that generally includes administering to a subject having or at risk of having cerebral ischemia-reperfusion injury an amount of any embodiment of the composition summarized above that is effective to ameliorate at least one symptom or clinical sign of cerebral ischemia-reperfusion injury.

In another aspect, this disclosure describes a method that generally includes administering to a subject having or at risk of having hypoxia an amount of the composition of any preceding claim effective to ameliorate at least one symptom or clinical sign of hypoxia. In some of these embodiments, the subject can have, or be at risk of having, acute mountain sickness (AMS).

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Structures and metabolism of AR isoflavones. Glycoside forms of isoflavones (calycosin-7-O-β-glucoside and ononin) are biologically inactive and can be transformed to non-glycoside forms (calycosin (C) and formononetin (F)) after absorption. F is then demethylated by intestine microbe to produce daidzein (D).

FIG. 2. Isoflavones reduced rates of cell death in primary cultured neurons under OGD/RO or L-glutamate-challenges. Rate of cell death was determined with LDH release assay. Isoflavones including calycosin (C), formononetin (F) and daidzein (D) were added into cultured media at the indicated concentrations prior to OGD/RO or L-glutamine (Glu) exposures. (A) Primary cultured neurons were exposed to four hours OGD followed by 20 hours reoxygenation. (B) Neurons were treated with 8 mM L-glutamate for 6 hours. Data were represented as Mean±SD, n=4, (*p<0.05, **p<0.01, compared to vehicle control).

FIG. 3. Combined isoflavone treatments had synergetic neuroprotective effects against OGD/RO and L-glutamine-induced cell death. Rates of cell death were determined with LDH release assay. Individual or combined isoflavones including calycosin (C), formononetin (F) and daidzein (D) were added into cultured media prior to OGD/RO (four hours OGD plus 20 hours RO) or L-glutamine exposures (8 mM L-glutamate for six hours). (A) Neurons treated with single isoflavone (2.5 μM) or the combined isoflavones (C2.5 μM/F2.5 μM, C2.5 μM/D2.5 μM, F2.5 μM/D2.5 μM, C2.5 μM/F2.5 μM/D2.5 μM). Data were represented as mean±SD, n=3, *p<0.05. (B) In L-glutamine experiments, primary cultured neurons were treated with different combinations of isoflavones prior to challenging by 8 mM L-glutamate for six hours. Data were represented as mean±SD, n=4, **p<0.01.

FIG. 4. Combined isoflavones groups synergistically induced Akt phosphorylation and inhibited cell death against OGD/RO and L-glutamine-induced cell death via ER-PI3K-Akt pathway. (A) Primary cultured neurons (DIV10) were incubated with 1 μM of ER antagonist ICI 182780 for 15 minutes before exposed to OGD/RO challenge (four hours OGD plus 20 hours RO) and treated with 25 μM isoflavones treatment. Rate of cell death was determined by LDH release assay. (B) Primary cultured neurons (DIV10) were incubated with 1 μM of ER antagonist ICI 182780 for 15 minutes before exposed to L-glutamate challenge (8 mM L-glutamate for six hours) and treated with 25 μM isoflavones treatment. Rate of cell death was determined by LDH release assay. (C) ER antagonist was added to the groups of combined isoflavones (total concentration 5 μM) following OGD/RO challenge. (D) ER antagonist was added to the groups of combined isoflavones (total concentration 5 μM) following L-glutamate challenge. (E) Western blot results in the expression of p-Akt and Akt. (F) Rates of cell death. Cells were pre-treated with 10 μM LY-294002 15 minutes before isoflavones treatment in L-glutamate challenging model. Cell lysate was collected for the Western analysis to detect the pAkt, whereas cell medium was used to determine the rate of cell death with LDH release assay. Data were represented as mean±SD, n=3, *p<0.05, n.s.: not significant.

FIG. 5. Brain infarct volume and neurology scores in rats subjected to focal ischemia and reperfusion with or without treatment of isoflavones and ER antagonist. (A) Brain infarct volume of each rat was assessed by 2,3,5-triphenyltetrazolium chloride (TTC) staining. (B) The infarct volume was evaluated by calculating the hemispheric lesion area with ImageJ software and presented as bar graph. (C) Neurology score is the sum of scores of dysfunctional paw test, postural reflex test and circling test. Data were represented as mean±S.D., n=6 (*p<0.05). (D-E) Rats were intracerebroventricularly injected with ICI 182780 one hour and intraperitoneally treated with isoflavones 30 minutes, respectively, before onset of ischemia. 24 hours later, rat brains were stained with TTC and quantified (*p<0.05, n=5).

FIG. 6. The infarct volume of the brains from MCAO rats received single or combined isoflavones treatment with or without PI3K inhibitor treatment. (A) The representative picture of infarct of brain sections with different isoflavones combinations treatment. (B) Infarct size in each group was calculated with ImageJ. (C) Images of infarct of rat brain section after intracerebroventricular injection with either LY294002 or vehicle control 60 minutes before onset of ischemia in C/F/D-mediated synergistic neuroprotective model. (D) Infarct size in rat brain section after intracerebroventricular injection with either LY294002 or vehicle control 60 minutes before onset of ischemia in C/F/D-mediated synergistic neuroprotective model. Data were represented as mean±SD, n=4-6, *p<0.05.

FIG. 7. GSFCD could scavenge peroxynitrite. HKYellow-AM staining of peroxynitrite treated with or without 50 μM synthesized peroxynitrite and GSFCD (1:1:1:1:1, 5 μM each of ginsenoside rg1, sodium danshensu, formononetin (F), calycosin (C), and daidzein (D)) for one hour. Scale bar=20 μm.

FIG. 8. GSFCD protected SH-SY5Y cells from OGD/RO-induced cell death. SH-SY5Y cells were pre-treated with GSFCD for 10 minutes, and then exposed to six hours OGD. Cells were retrieved from the chamber and returned to normal medium for reoxygenation, Cells were exposed to six hours OGD followed by four hours reoxygenation. Cell viability was assessed by XTT assay. **p<0.01, compared to normoxia group; ##p<0.01, compared to OGD/RO group.

FIG. 9. GSFCD protected neurons from OGD/RO-induced cell death. (A) Neurons were pretreated with GSFCD for 10 minutes, and then cells were subjected to OGD four hours, followed by 20 hours reperfusion. XTT assay was performed to assess cell viability. **p<0.01, compared to normoxia group; ^{##}p<0.01 compared to OGD/RO group. (B) TUNEL staining and DAPI staining of each group. (C) The percentage of cell death was computed from TUNEL staining data. **p<0.01, compared to normoxia group; ^{##}p<0.01 compared to OGD/RO group. Scale bar=20 μm.

FIG. 10. GSFCD protected rat brains from cerebral ischemia-reperfusion injury. (A) Representative brain coronal section image showing infarct volume as visualized by TTC staining. Red-colored regions indicate non-ischemic and white regions indicate ischemic. (B) Quantification of infarct volume (% of whole brain volume) by image J (n=5). **p<0.01, compared to MCAO vehicle group.

FIG. 11. Chemical structures. (A) Ginsenoside Rg1; (B) Sodium danshensu.

### DETAILED DESCRIPTION OF ILLUSTRATIVE EMBODIMENTS

This disclosure describes, in one aspect, a composition that includes calycosin, formononetin, and a pharmaceutically acceptable carrier. The composition may be formulated into a clinical agent for treating, for example, ischemic stroke and/or as an over-the-counter neutraceutical for ameliorating at least one symptom or clinical sign of, for example, high mountain altitude syndrome.

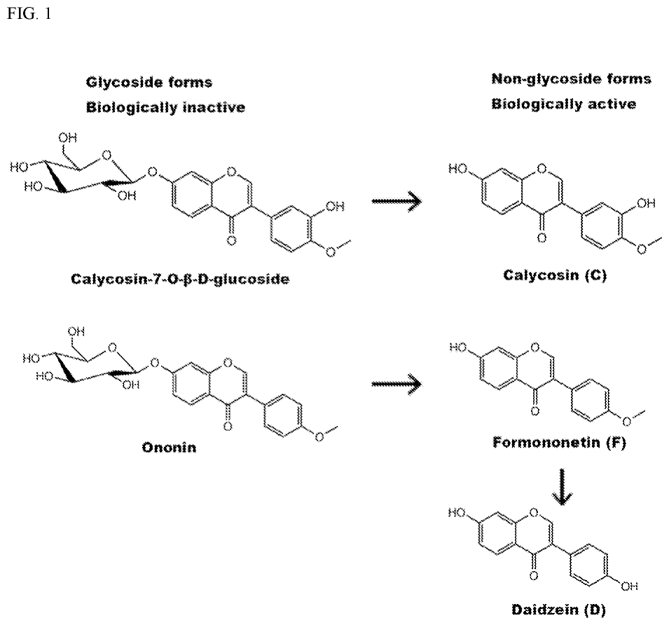

Astragali Radix (AR) is the dried root of Astragalus membranaceus (Fisch.) Bunge and Astragalus mongholicus Bunge (Fabaceae). AR contains isoflavones that have been used as food supplements due to their antioxidant properties. There are four isoflavones in AR: calycosin (C) and formononetin (F) as well as two glycoside form C-glycoside and F-glycoside (Ononin). Glycoside forms of isoflavones are biologically inactive but they can be transformed to non-glycoside forms after absorption (FIG. 1). Through demethylation by intestinal microbes, F can be transformed into daidzein (D). C, F, and D represent the total functional AR isoflavones after absorption, and therefore, were selected for study. All three isoflavones can bind to estrogen receptors (ER) and exert their estrogenic effects as “phytoestrogens” although their binding affinities and transactivation capabilities are relatively lower than 17β-estradial. Calyscosin-7-O-β-D-glucoside, a glucoside form of C in AR, affords neuroprotective effects against ischemic brain injury through non-canonical pathway of ER signaling and harboring anti-oxidative capability. F can protect against cerebral ischemia-reperfusion injury. It is of great interest to determine whether these isoflavones, each of which is active individually, can act synergistically to afford greater effect against cerebral injury due to ischemia-reperfusion injury and/or hypoxia.

The present study was designed to determine whether AR isoflavanes have synergic effects against cerebral ischemia-reperfusion injury through activating ER-phosphoinositide 3-kinase (PI3K)-Akt pathway. The neuroprotective effects of C, F, and D—both individually and synergistically—against cerebral hypoxia and ischemia-reperfusion injury were investigated using in vitro primary cultured oxygen-glucose deprivation (OGD) neurons and in vivo cerebral ischemia-reperfusion rat models. The combination of C, F, and D yielded much greater neuroprotective effects than the sum of each individual isoflavone, indicating the synergistic neuroprotective effects of different isoflavones contributing to the neuropharmacological effects of AR.

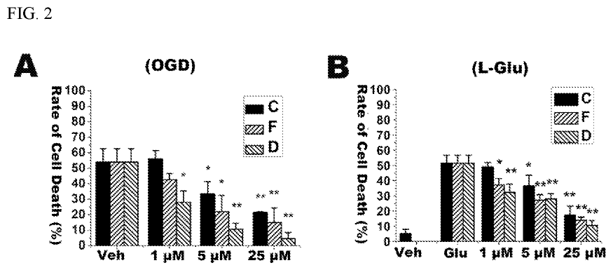

First, the neuroprotective effects of C, F, and D on primary cultured cortical neurons were investigated under OGD/RO conditions (hypoxia and stroke cellular model) or L-glutamine challenge (excitotoxicity cellular model). In OGD experiments, primary cultured cortical neurons were subjected to four hours OGD followed by 20 hours RO and the rates of cell death were determined. FIG. 2 shows that each isoflavone individually had a dose-dependent inhibitory effect against OGD/RO-induced cell death and L-glutamate-induced cell death, indicating that each isoflavone has neuroprotective effects.

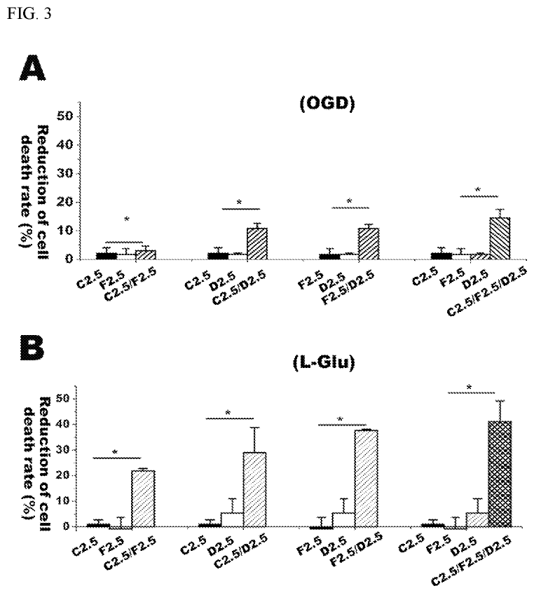

Moreover, isoflavones exhibit synergistic effects inhibiting OGD/RO-induced cell death and L-glutamate-induced cell death in vitro. The ratio of C and F (including glucoside conjugates) in AR has been reported to be 1:1, whereas D was undetectable in raw materials. Pharmacokinetics studies revealed that these isoflavones were present in serum and that about 50% of F was metabolized into D and its conjugates after an AR extract was orally administrated to rats. Thus, experiments were performed to determine whether these isoflavones have synergistic neuroprotective effects against OGD/RO-induced neuronal cell death and/or L-glutamate-induced neuronal cell death. Various experimental groups were treated with a single isoflavone or a combination of isoflavones. Single flavone compositions included C2.5 (2.5 μM calycosin), F2.5 (2.5 μM formononetin), and D2.5 (2.5 μM daidzein). Combined isoflavone compositions included C2.5/F2.5 (2.5 μM calycosin+2.5 μM formononetin), C2.5/D2.5 (2.5 μM calycosin+2.5 μM daidzein), F2.5/D2.5 (2.5 μM formononetin+2.5 μM daidzein) and C2.5/F2.5/D2.5 (2.5 μM calycosin+2.5 μM formononetin+2.5 μM daidzein). Primary cultured neurons were pre-treated with an isoflavone composition (single isoflavone or combination) prior to OGD/RO or L-glutamate treatments. The reduction of cell death rate as compared to vehicle control was determined. In the OGD/RO experiments, single isoflavone (C, F, or D) treatments exhibited only a 2% reduction in the rate of cell death whereas the C/D combination and the D/F combination exhibited about a 10% reduction in the rate of cell death and the C/D/F combination exhibited a 15% reduction in the rate of cell death, indicating the synergistic effects of those isoflavones against OGD/RO-induced cell death (FIG. 3A).

The synergistic effects of isoflavones also were found in L-glutamate-challenged experiments. Exposure of 8 mM L-glutamate for six hours induced about 45% of cell death. Individual isoflavone treatment (C, F, D) at 2.5 μM exhibited less than a 2% reduction in the rate of cell death while the C/F combination exhibited a 22% reduction in the rate of cell death, the C/D combination exhibited a 28% reduction in the rate of cell death, and the D/F combination exhibited a 40% reduction in the rate of cell death. The C/D/F combination exhibited a 42% reduction in the rate of cell death (FIG. 3B). These results strongly indicate that AR isoflavones have synergistic neuroprotective effects against OGD/RO and L-glutamate-induced cell death.

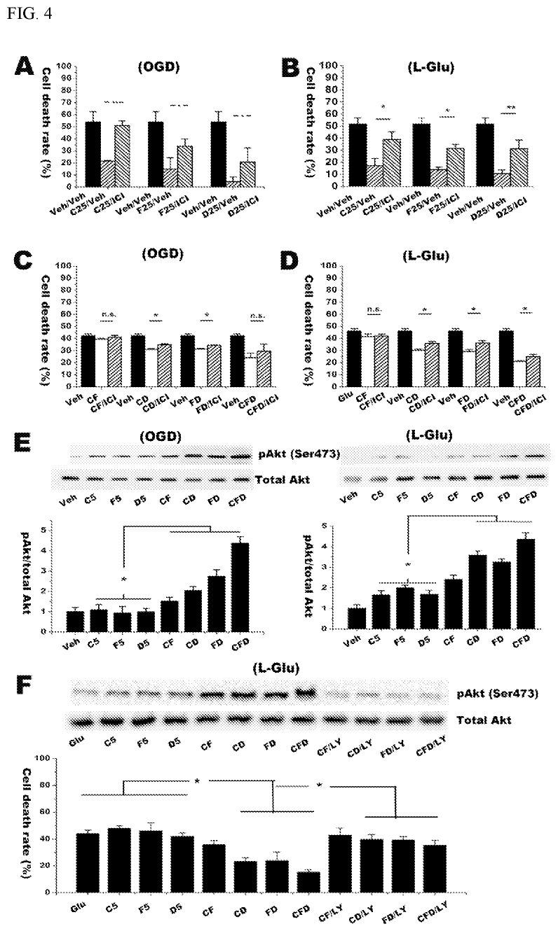

Isoflavones also have synergistic effects inducing ER-PI3K-Akt pathway in vitro. The synergistic neuroprotective effects of isoflavones involve activating non-classical ER signaling. FIG. 4A and FIG. 4B show that pre-treatment with ICI 182780 decreased the neuroprotective effects of each individual isoflavone compositions in both OGD/RO and L-glutamate challenging models. ICI 182780 pre-treatment also decreased the synergistic neuroprotective effects of the isoflavone combinations (the sum of each isoflavones were 5 μM, FIG. 4C and FIG. 4D).

Levels of pAkt Ser473 were assessed in each experimental group. Estrogen receptors located at or near membrane are activated, followed by a rapid response that involves activating downstream cell survival signaling such as PI3K/Akt and participates in neuroprotection in cerebral ischemia induced neuronal insults. Activated PI3K can induce phosphorylation of Akt at Ser473. FIG. 4E shows that pre-treatment with a combination of isoflavones had significantly higher phosphorylation level of Akt at Ser473 than pre-treatment with a single isoflavone. This result was seen in both the L-glutamate model and the OGD/RO model. The combination of all three isoflavones yielded the highest pAkt expression level. Moreover, PI3K inhibitor LY-294002 was used to verify the role of pAkt in the synergistic effects of AR isoflavones. Results showed that pretreatment of LY-294002 (10 μM) significantly down-regulated the expression of pAkt. LDH release assay revealed that LY-294002 reserved the effects of the combined C/D, F/D and C/F/D treatments in L-glutamate-mediated neurotoxicity (FIG. 4F). These data suggest that the isoflavones synergistically protect neurons under the challenge of L-glutamate through PI3K/Akt pathway.

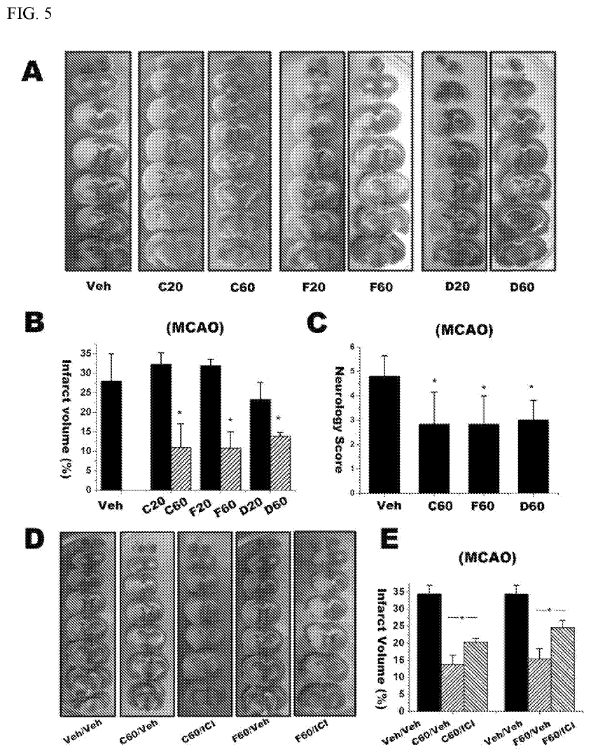

AR isoflavones decreased infarct volume and improved neurological outcome in rat MCAO ischemia-reperfusion model. The neuroprotective effects of these isoflavones were examined by using in vivo MCAO cerebral ischemia-reperfusion model. After the rats were subjected to 1.5 hours MCAO plus 22.5 hours reperfusion, vehicle treatment group revealed infarct volume up to 27.93%±7.1%. In three isoflavones treatment groups, rats treated with the dose of 60 μmol/kg C (17.06 mg/kg), F (16.1 mg/kg), or D (15.3 mg/kg) exhibited smaller infarct volumes. Rats receiving C treatment exhibited an infarct volume of 10.99±6.07%, rats receiving F treatment exhibited an infarct volume of 10.76±4.21%, and rats receiving D treatment exhibited an infarct volume of 13.84±0.95% (FIG. 5A and FIG. 5B). Lower dosage of each isoflavone (20 μmol/kg, equivalent to 5.69 mg/kg for C, 5.37 mg/kg for F, and 5.1 mg/kg for D) treatment had no statistical difference in comparison with the vehicle group (P>0.05). Functional recovery based on behavior deficit is a major endpoint in clinical trials. In neurological deficit scores, rats treated with 60 μmol/kg C, 60 μmol/kg F, or 60 μmol/kg D displayed much improved neurological function than vehicle group (FIG. 5C).

Next, the ability of ER antagonist ICI 182780 to block the synergic neuroprotective effects of isoflavones was examined. Rats were intracerebroventricularly injected with ICI 182780 at one hour before undergoing cerebral FR. TTC staining picture (FIG. 5D) and quantitative data (FIG. 5E) showed that co-treatment of ICI 182780 partially blocked the neuroprotective effects of C and F on infarct volume. These in vivo animal experimental results are consistent with in vitro cellular experiments.

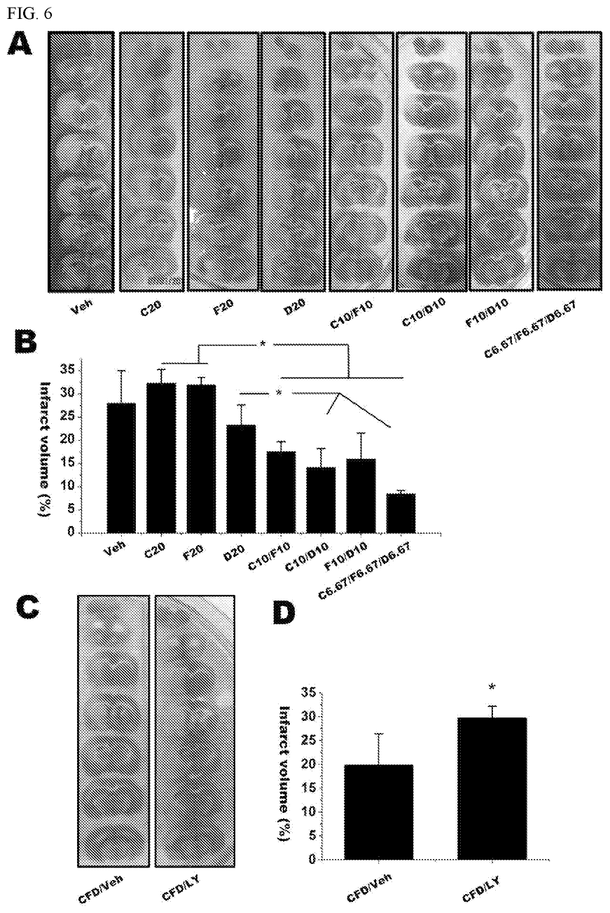

Isoflavones synergistically protect against cerebral ischemia-reperfusion injury via activating PI3K-Akt signaling pathway. Next, the synergistic effects of the isoflavones in reducing infarction volume in the in vivo MCAO model were investigated with the same protocol. FIG. 6A and FIG. 6B show that low doses of isoflavones had no significant effect on reducing infarction volume. Rats were treated with 20 μmol/kg of C (C20, equivalent 5.69 mg/kg), F (F20, equivalent 5.37 mg/kg), or daidzein (D20, equivalent 5.1 mg/kg) had no significant effect on reducing infarction volume. Rates treated with a combination of isoflavones, such as C10/F10 (10 μmol/kg C+10 μmol/kg F), significantly reduced infarct sizes in comparison with treatment of C or F alone in the dosage of 20 μmol/kg (C20 or F20). Furthermore, the combined treatment of C, F and D (6.67 μmol/kg of each) showed the best neuroprotective effects among all groups. These results suggest that combined isoflavones formula synergistically decreased infarct volume in rat brains after MCAO ischemia-reperfusion.

Next, the ability of AR isoflavones to synergistically regulate the PI3K-Akt signaling pathway was examined using the PI3K inhibitor LY-294002. LY-294002 (CFD-LY) was intracerebroventricularly injected into the rats before the combined C/F/D treatment. FIG. 6C and FIG. 6D show that pretreatment of LY-294002 significantly reduced the effects of combined C/F/D treatment on infarct volume. These results indicate that AR isoflavones have the synergistic neuroprotective effects through activating PI3K/Akt signaling pathway.

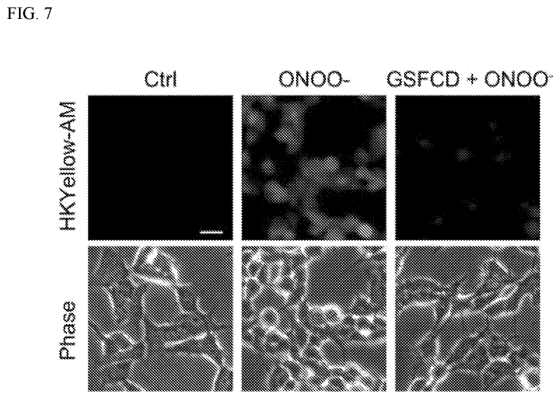

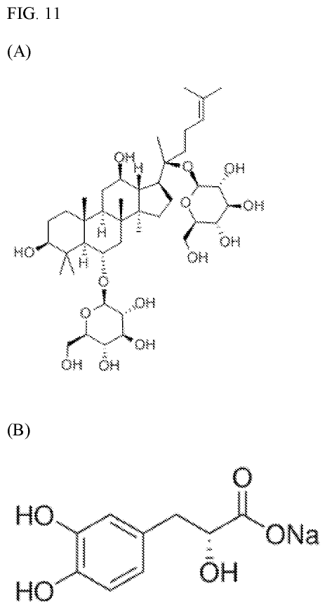

Neuroprotection was also observed using a 1:1:1:1:1 mixture of ginsenoside rg1 (FIG. 11A), sodium danshensu (FIG. 11B), formononetin (F), calycosin (C), and daidzein (D). The combination (GSFCD) scavenged peroxynitrite in SH-SY5Y cells using synthesized authentic peroxynitrite. HKYellow-AM (a novel specific peroxynitrite fluorescent probe) staining, which allows one to visualize peroxynitrite level in vitro. GSFCD reduced synthesized authentic peroxynitrite fluorescent signaling (FIG. 7). The data suggested that GSFCD could efficiently scavenge peroxynitrite in SH-SY5Y cells. Ginsenosides have antioxidant properties. Danshensu suppresses the formation of reactive oxygen species, inhibits platelet adhesion and aggregation, and protects against ischemic injury. Neither ginsenosides nor danshensu are found in Astragali Radix (AR). Adding G and S into the formula that already includes C, F, and D therefore increases the efficacy and potency of the formula as a treatment for ischemic stroke and/or a “nutraceutical” targeting hypoxia brain injury (e.g., high mountain altitude syndrome).

While described above in the context of a particular embodiment in which a ginsenoside and danshensu are added to the formulation, the formulation can include either one of the compounds without the other. Moreover, the formulation can include any compound having antioxidant properties in place of or in addition to a ginsenoside and/or danshensu.

The ability of GSFCD to inhibit ischemia-reperfusion injury and hypoxia brain injury was assessed in vitro through OGD/RO experiments. SH-SY5Y cells were subjected to six hours of OGD followed by four hours of reoxygenation. Results showed that GSFCD protected cells from OGD/RO-induced cell death (FIG. 8).

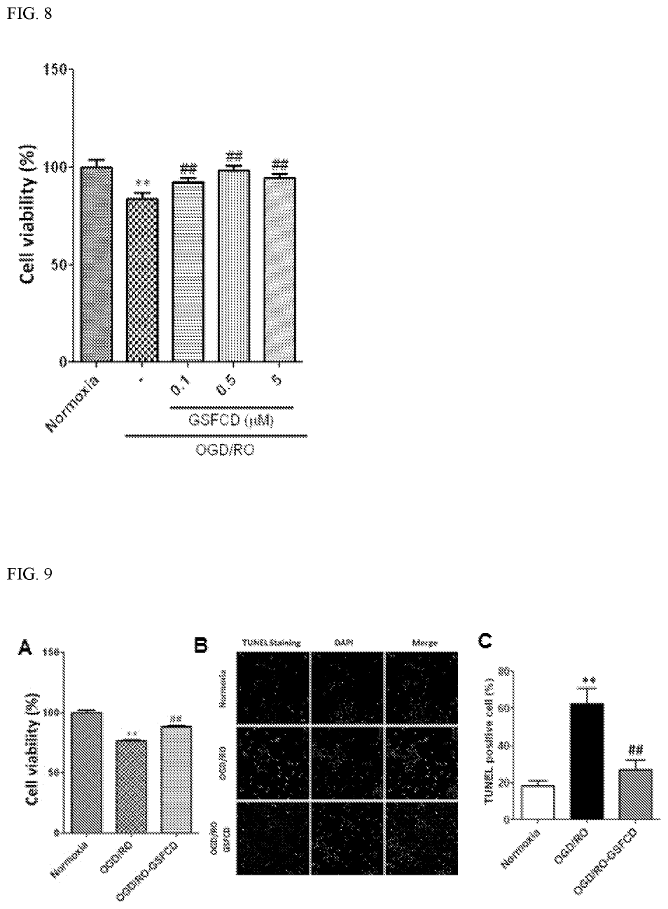

GSFCD also protected primary neurons from OGD/RO-induced cell death. The neuroprotective effects of GSFCD were confirmed in primary cultured rat neuron. GSFCD also showed protective effects in neurons under OGD/RO condition (FIG. 9).

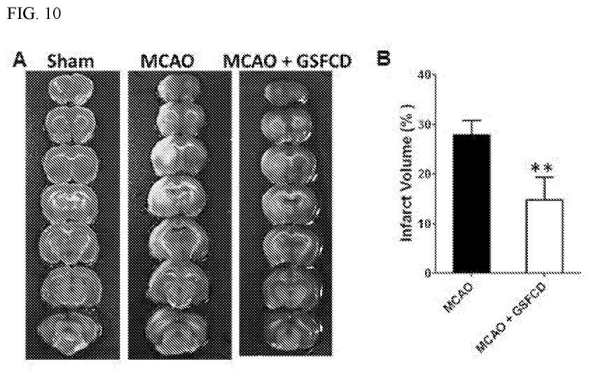

Finally, GSFCD protected rat brain against cerebral ischemia-reperfusion injury. Adult SD rats were subjected to MCAO model with two hours ischemia followed by 22 hours reperfusion. GSFCD (1:1:1:1:1, 10 μmol/kg, total:18.27 mg/kg) was intraperitoneally injected into rats at the onset of the reperfusion stage. GSFCD treatment remarkably reduced infarct volume in ischemia-reperfused brain (FIG. 10).

This is the first report demonstrating the synergistic neuroprotective effects of isoflavones with similar parent structure on cerebral ischemia-reperfusion injury. Furthermore, our results revealed that AR isoflavones synergistically regulate ER-PI3K-Akt signaling pathway and subsequently protect neurons from cerebral ischemia-reperfusion injury.

Astragali Radix is a representative herb used for stroke treatment. Isoflavones are its representative neuroprotective ingredients. The combined formula with C/D, D/F or C/D/F exhibited increased neuroprotective effects compared with individual isoflavones at the same doses in the in vitro models of OGD/R-induced neuronal injury and L-glutamate-induced neuronal injury, and in vivo rat model of MCAO cerebral ischemia and reperfusion. These results suggest that the combined drug strategy provides superior activity compared to a single drug therapeutic approach for improving therapeutic outcome in ischemic stroke treatment.

The neuroprotective effects of individual isoflavones and combined AR isoflavones were decreased by ER antagonist and PI3K inhibitor. This study, for the first time, describes the contribution of the ER signaling pathway to isoflavones-mediated neuroprotective effects and in enriching the mechanisms of the beneficial effects of AR.

The ER-PI3K-Akt pathway may partly explain the mechanisms of the synergistic roles of AR isoflavones. However, how these isoflavones synergistically work together is still unclear. Several possible mechanisms could contribute to the observed synergism. First, isoflavones could bind to ER with different binding affinity, but the binding sites are unclear yet. It is possible that binding of one isoflavone might change the ER structure state and increase the binding affinity to other isoflavone, which is an allosteric effect. Second, isoflavone may increase the expression of ER. Phytoestrogens might alter the expression of estrogen receptors. For example, mice fed with genistein showed increased expression of ERα in ovary, D can increase mRNA and protein expression of ERα and ERβ in granulosa cells, and perinatal mice treated with daidzein display elevated levels of ERα expression in brains. Thus, C, F, and/or D might increase ER expression while elevated expression would produce more binding sites for isoflavone molecules and thereby produce the synergistic neuroprotective effects. Third, isoflavones have anti-oxidative and/or iron-binding effects. Although isoflavones are known to have abilities of scavenging free radicals and ER transactivation, their bioactivities might be different, potentially contributing to the synergism when they work together. Further study is required to elucidate the exact mechanism of the synergism.

In conclusion, AR isoflavones synergistically protect neurons against ischemia-reperfusion injury in vivo and in vitro and the ER-PI3K-Akt pathway is likely the molecular target of AR isoflavones contributing to their synergistic neuroprotection. This study provides a new cue for understanding the neuroprotective role of AR and its related formula.

Accordingly, this disclosure describes compositions that include two or more isoflavones in combination so that the combination provides a synergistic neuroprotective property compared to the additive effect of the isoflavones if administered alone. Generally, the composition includes at least two isoflavones and a pharmaceutically acceptable carrier. The composition described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the isoflavones, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the isoflavones without causing any undesirable biological effects or interacting in a deleterious manner with any of the other components of the pharmaceutical composition in which it is contained.

The isoflavones may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, the isoflavones may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the isoflavones into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of isoflavones administered can vary depending on various factors including, but not limited to, the specific isoflavone compounds, the weight, physical condition, and/or age of the subject, and/or the route of administration. Thus, the absolute weight of isoflavones included in a given unit dosage form can vary widely, and depends upon factors such as the species, age, weight and physical condition of the subject, and/or the method of administration. Accordingly, it is not practical to set forth generally the amount that constitutes an amount of isoflavones effective for all possible applications. Those of ordinary skill in the art, however, can readily determine the appropriate amount with due consideration of such factors.

In some embodiments, the method can include administering sufficient isoflavones to provide a dose of, for example, from about 100 ng/kg to about 100 mg/kg to the subject, although in some embodiments the methods may be performed by administering the isoflavones in a dose outside this range. In some of these embodiments, the method includes administering sufficient isoflavones to provide a minimum does of at least 1 μg/kg such as, for example, a dose of at least 10 μg/kg, at least 50 μg/kg, at least 100 μg/kg, at least 200 μg/kg, at least 300 μg/kg, at least 400 μg/kg, or at least 500 μg/kg. In some embodiments, the method includes administering sufficient isoflavones to provide a maximum does of no more than 100 mg/kg such as, for example, a dose of no more than 50 mg/kg, no more than 10 mg/kg, no more than 5 mg. kg, or no more than 1 mg/kg. In some embodiments, the method can include administering an amount of isoflavones defined as a range having as endpoints any minimum dose listed above and any maximum dose listed above that is greater than the minimum dose. In particular embodiments, for example, the method can include administering isoflavones in a dose of from 1 μg/kg to 100 mg/kg, from 10 μg/kg to 10 mg/kg, or from 500 μg/kg to 5 mg/kg.

Accordingly, a pharmaceutical composition can include isoflavones provided at a concentration suitable to provide the desired dosage. For example, each isoflavone present in a pharmaceutical composition can be provided, independently of the concentration of other isoflavones in the composition, at a concentration of at least 1 μM such as, for example, at least 2 μM, at least 5 μM, at least 10 μM, or at least 20 μM. In particular embodiments, the concentration of an isoflavone, independent of the concentration of other isoflavones in the composition, can be 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, or 20 μM.

In some embodiments, the isoflavones may be administered, for example, from a single dose to multiple doses per day, although in some embodiments the method can be performed by administering the isoflavones at a frequency outside this range. In certain embodiments, the isoflavones may be administered from about once per month to multiple times per day such as, for example, once per week, once per day, or three times per day.

In some embodiments, the isoflavones may be administered prophylactically or, alternatively, can be initiated after the subject exhibits one or more symptoms or clinical signs of ischemic stroke or hypoxia. Treatment that is prophylactic is referred to herein as treatment of a subject that is “at risk” of having the condition. For example, an isoflavone composition may be used to prophylactically treat hypoxia—e.g., acute mountain sickness (AMS)—before traveling to a high-altitude destination and consequently, before the effects of high altitude manifest in the subject.

Thus, as used herein, the term “at risk” refers to a subject that may or may not actually possess the described risk. Thus, for example, a subject “at risk” of developing hypoxia (e.g., acute mountain sickness (AMS)) is a subject present in, or travelling to, an area where other individuals have developed hypoxia under similar circumstances and/or otherwise possess one or more risk factors associated with the hypoxia such as, for example, genetic predisposition, ancestry, age, sex, geographical location, lifestyle, or medical history, even if the subject does not manifest any symptom or clinical sign of hypoxia.

Accordingly, a composition can be administered before, during, or after the subject first exhibits a symptom or clinical sign of the condition. Treatment initiated before the subject first exhibits a symptom or clinical sign associated with the condition—e.g., hypoxia—may result in decreasing the likelihood that the subject experiences clinical evidence of the condition compared to a subject to which the isoflavone composition is not administered, decreasing the severity of symptoms and/or clinical signs of the condition, and/or completely resolving the condition. Treatment initiated after the subject first exhibits a symptom or clinical sign associated with the condition—e.g., ischemic stroke or hypoxia—may result in decreasing the severity of symptoms and/or clinical signs of the condition compared to a subject to which the composition is not administered, and/or completely resolving the condition.

Thus, the method includes administering an effective amount of the composition to a subject having, or at risk of having, a particular condition. In this aspect, an “effective amount” is an amount effective to reduce, limit progression, ameliorate, or resolve, to any extent, a symptom or clinical sign related to the condition.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Drugs and Reagents

Isoflavones including C, F, and D isolated from crude extract of AR were obtained from Forever Biotech Inc., Shanghai, China. The purity of AR isoflavones was determined with HPLC to be at >98% for C and F, and >99% for D.

### Primary Cell Culture

Primary cultured cortical neurons were isolated from embryonic Sprague-Dawley rats (E17). After digested by 0.25% trypsin (Invitrogen Corp., Carlsbad, Calif.), cells were collected and suspended in Dulbecco's modified eagle medium (DMEM) containing 10% horse serum, 1% L-glutamine and antibiotic mixture. The resulting single cell suspensions were seeded on 24-well or 6-well plates pre-treated with poly-D-lysine (Sigma-Aldrich, St. Louis, Mo.). After seeded for four hours, medium was replaced with neurobasal medium containing 2% B27, 1% L-glutamine and antibiotics mixture and cell culture was maintained at 37° C. and 5% CO₂. Cell purity was found to be more than 95% by using immunostaining of Tuj-1 (1:200, Santa Cruz Biotechnology, Dallas, Tex.), a marker of mature neuron.

### In Vitro Experiments and Drug Treatment

Both oxygen-glucose deprivation plus reoxygenation (OGD/RO) and excitotoxicity experiments were conducted. In OGD/RO experiments, primary cultured neurons were exposed to neurobasal medium without glucose and incubated in a hypoxia chamber (Billups-Rothenberg, Inc., San Diego, Calif.) perfused with a mixed gas containing 95% N₂ and 5% CO₂ until medium oxygen concentration reached 0.1%, which was monitored with a PA-10A paramagnetic 02 analyzer (Sable Systems International, North Las Vegas, Nev.). After cells were exposed to OGD at 37° C. for four hours, the medium was replaced with normal glucose-containing medium and the plates were put into normal culture condition for 20 hours for reoxygenation. In excitotoxicity experiments, primary cultured neurons were treated with L-glutamate (8 mM, Sigma-Aldrich, St. Louis, Mo.) for six hours. Different dosages (1 μM, 5 μM, and 25 μM) of C, F, and D were added to cultured medium at 15 minutes prior to OGD/RO or L-glutamate exposure.

### Lactate Dehydrogenase (LDH) Release Assay for Detecting Cell Toxicity

LDH release assay was performed for detecting cell death with Cytotoxicity Detection Kit (Invitrogen Corp., Carlsbad, Calif.). In brief, culture medium was collected and incubated with the reaction mixture for 30 minutes at room temperature (24° C.). Cell-free medium collected in the well was used as negative control counting as zero percent. Triton-X 100-treated cell medium was used as 100% cell death control. The optical density of the solution was measured at 490 nm on a microplate reader (Model 3350, Bio-Rad Laboratories, Inc., Hercules, Calif.). The cell death rate was calculated by using the formula: Cell death rate (%)=(Experimental absorbance value−culture medium absorbance value)/(Triton-X 100-treated absorbance value−culture medium absorbance value)×100%.

### Middle Cerebral Artery Occlusion (MCAO) Model and Drug Treatments

Male adult Sprague-Daweley rats (250 g to 270 g) were obtained from Laboratorial Animal Unit, the University of Hong Kong (HKU) and University of New Mexico (UNM). All animal experiments were approved and regulated by the Committee on the Use of Live Animals in Teaching and Research, HKU and the Laboratory Animal Care and Use Committee, UNM. All efforts were made to minimize animal suffering and to reduce the number of animals used.

Rats were subjected to MCAO to induce cerebral ischemia-reperfusion as previously described (Shen et al., 2006. J Neurochem 96(4):1078-1089). Briefly, rats were anaesthetized by inhalation of 5% isoflurane and maintained with 2% isoflurane in a mixture of 70% N₂O and 30% 02. After vessels isolation, a 3/0 monofilament nylon suture (Johnson & Johnson Health Care Systems, Inc., Piscataway, N.J.) was inserted into the external carotid artery and advanced into the internal carotid artery and anterior cerebral artery to occlude the middle cerebral artery. After occlusion for 1.5 hours, the suture was removed to induce reperfusion and maintained for 22.5 hours. After operation, rats were transferred to intensive care incubator in which the temperature was kept at 37° C. until animals woke up completely.

Isoflavones (C, F, and D) were dissolved in dimethyl sulfoxide (DMSO) and diluted with peanut oil to the indicated working solution and intraperitoneally (i.p.) injected to rats 30 minutes before suture insertion for MCAO. The same volume of DMSO and peanut oil were used as vehicle control. In parallel experiments, for mechanistic study, ER antagonist ICI 182780 or PI3K inhibitor LY-294002 was intracerebroventricularly (i.c.v.) injected into the rat lateral ventricle 60 minutes before ischemia onset. Briefly, after anaesthetization, rat was placed on a stereotaxic apparatus and fixed well. A 22-gauge, 12 mm stainless-steel guide cannula was inserted in the left lateral ventricle of the brain. The needle was inserted 4.5 mm deep, 1.5 mm lateral to sagiture and 0.8 mm posterior to bregma to reach the lateral ventricle. Then, ICI 182780 dissolved in DMSO (1 μM) or LY-294002 (10 mM) was slowly injected into lateral ventricle and the needle was kept for at least 30 seconds before removal. The same volume of DMSO was used as a negative control.

### Measurement of Infarct Volume and Neurology Score

After 24 hours of reperfusion, rat brains were isolated and sliced into coronal sections followed by staining with 2,3,5-triphenyltetrazolium chloride (TTC, Sigma-Aldrich, St. Louis, Mo.) at 37° C. for 20 minutes. The ischemic area was evaluated by calculating the hemispheric lesion area with ImageJ software (ImageJ-1.38x; Schneider et al., 2012. Nature methods 9(7):671-675), the relative infarct volume percentage (RIVP) was calculated as RIVP=IVA/TA×100%, IVA was the total infract area of five coronal sections, TA was the total area of five sections.

Neurological function was determined by 9-scale methods containing dysfunctional paw test, postural reflex test and circling test according to a previous report (Bederson et al., 1986. Stroke 17(3):472-476). Each score was summed and represented as a single overall neurological score (0 to 8).

### Western Blot Analysis

Denatured protein samples were resolved on SDS-PAGE and transferred to PVDF membrane (Millipore Corp., Billerica, Mass.). After blocking, membrane was incubated overnight at 4° C. with antibodies including pAkt (Ser473) (1:500, Cell Signaling Technology, Inc., Danvers, Mass.), Akt (1:1000, Cell Signaling Technology, Inc., Danvers, Mass.), GAPDH (1:5000, Cell Signaling Technology, Inc., Danvers, Mass.). After washing, the membrane was incubated with the goat anti-mouse or anti-rabbit HRP-conjugated secondary antibodies (1:2000, Santa Cruz Biotechnology Inc., Dallas, Tex.). Chemiluminescence detection was performed using ECL advance western blotting detection reagents (GE Healthcare, Little Chalfont, UK).

### Statistical Analysis

Data were expressed as Means±Standard Deprivation (SD). For multiple groups designed experiments, comparisons were made by one-way analysis of variance (ANOVA) and followed by Dunnett test for two group comparisons within the multiple groups. For two groups designed experiments, comparisons were determined using unpaired Student's t-test. Statistical analysis was performed in the SPSS 16.0 statistical program (IBM Corp., Armonk, N.Y.) in which p<0.05 was considered to be statistically significant.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A composition comprising:
a first isoflavone compound;
a second isoflavone compound, different than the first compound;
each of the first isoflavone compound and the second isoflavone compound provided in an amount, in combination with the other isoflavone compound, effective to decrease infarct volume in a rat model; and
a pharmaceutically acceptable carrier.

2. A composition comprising:
a first isoflavone compound;
a second isoflavone compound, different than the first compound;
each of the first isoflavone compound and the second isoflavone compound provided in an amount, in combination with the other isoflavone compound, effective to decrease neural death by in vivo treatment with L-glutamate; and
a pharmaceutically acceptable carrier.

3. A composition comprising:
a first isoflavone compound;
a second isoflavone compound;
each of the first isoflavone compound and the second isoflavone compound provided in an amount, in combination with the other isoflavone compound, effective to decrease neural death in an in vivo oxygen-glucose deprivation plus reoxygenation (OGD/RO) model; and
a pharmaceutically acceptable carrier.

4. The composition of claim 1, wherein:
the first isoflavone compound is provided at a concentration of from 1 μM to 20 μM; and
the second isoflavone compound is provided at a concentration of from 1 μM to 20 μM.

5. The composition of claim 1, wherein:
the first isoflavone compound comprises calycosin, formononetin, or daidzein; and
the second isoflavone compound comprises calycosin, formononetin, or daidzein.

6. The composition of claim 1, further comprising a third isoflavone compound, different than the first compound and the second compound.

7. The composition of claim 6, wherein the third isoflavone compound comprises calycosin, formononetin, or daidzein.

8. The composition of claim 1, further comprising ginsenoside Rg1 in an amount effective to further decrease infarct volume in a rat model, further decrease neural death by in vivo treatment with L-glutamate, or further decrease neural death in an in vivo OGD/RO model.

9. The composition of claim 8, wherein the ginsenoside Rg1 is provided at a concentration of from 1 μM to 20 μM.

10. The composition of claim 1, further comprising a salt of danshensu in an amount effective to further decrease infarct volume in a rat model, further decrease neural death by in vivo treatment with L-glutamate, or further decrease neural death in an in vivo OGD/RO model.

11. The composition of claim 10 wherein the salt of danshensu is provided at a concentration of from 1 μM to 20 μM.

12. A method comprising:
administering to a subject having or at risk of having cerebral ischemia-reperfusion injury an amount of the composition of claim 1 effective to ameliorate at least one symptom or clinical sign of cerebral ischemia-reperfusion injury.

13. A method comprising:
administering to a subject having or at risk of having hypoxia an amount of the composition of claim 1 effective to ameliorate at least one symptom or clinical sign of hypoxia.

14. The method of claim 13, wherein the subject has, or is at risk of having, acute mountain sickness (AMS).

15. The composition of claim 2, wherein:
the first isoflavone compound comprises calycosin, formononetin, or daidzein; and
the second isoflavone compound comprises calycosin, formononetin, or daidzein.

16. The composition of claim 2, further comprising a third isoflavone compound, different than the first compound and the second compound.

17. The composition of claim 2, further comprising ginsenoside Rg1 in an amount effective to further decrease infarct volume in a rat model, further decrease neural death by in vivo treatment with L-glutamate, or further decrease neural death in an in vivo OGD/RO model.

18. The composition of claim 2, further comprising a salt of danshensu in an amount effective to further decrease infarct volume in a rat model, further decrease neural death by in vivo treatment with L-glutamate, or further decrease neural death in an in vivo OGD/RO model.

19. The composition of claim 3, wherein:
the first isoflavone compound comprises calycosin, formononetin, or daidzein; and
the second isoflavone compound comprises calycosin, formononetin, or daidzein.

20. The composition of claim 3, further comprising a third isoflavone compound, different than the first compound and the second compound.

21. The composition of claim 3, further comprising ginsenoside Rg1 in an amount effective to further decrease infarct volume in a rat model, further decrease neural death by in vivo treatment with L-glutamate, or further decrease neural death in an in vivo OGD/RO model.

22. The composition of claim 3, further comprising a salt of danshensu in an amount effective to further decrease infarct volume in a rat model, further decrease neural death by in vivo treatment with L-glutamate, or further decrease neural death in an in vivo OGD/RO model.
